(19) United States
(12) Patent Application Publication
(10) Pub. No.: US 2021/0283527 A1
(43) Pub. Date: Sep. 16, 2021

(54) PLANT MATTER SMOKE AND VAPOR COLLECTION DEVICE

(71) Applicant: Izun Pharmaceuticals Corporation

(72) Inventors: Shmuel Cohen, Nehosha (IL); William Z. LEVINE, Jerusalem (IL)

(73) Assignee: Izun Pharmaceuticals Corporation, New York, NY (US)

(21) Appl. No.: 16/332,377

(22) PCT Filed: Sep. 12, 2017
(86) PCT No.: PCT/IL2017/051030

§ 371 Date: Mar. 12, 2019

(51) Int. Cl. B01D 3/40; A61K 36/00

(52) U.S. Cl. CPC B01D 3/40; A61K 36/00; C07K 14/765; A61K 2236/50; A61K 2236/333

(57) ABSTRACT

Embodiments of the disclosure relate to a plant matter burning or vaporization apparatus comprising a plant matter receptacle for containing plant matter, and a burning/vaporization region for burning or vaporization of plant matter to produce a plant compound vapor, and a plant matter conduit operable to continuously convey plant matter to the burning/vaporization region. Embodiments of the disclosure also relate to a solvent combination apparatus comprising or operatively connected to a vapor conduit located at or at a distance from the burning/vaporization region, from which the plant compound vapor is removed from the environment of the plant matter and conveyed to a solvent in which the plant compound vapor is dissolved. The dissolution of the plant compound vapor in the solvent may be assisted by passing the solvent and smoke/vapor through a nozzle creating a region of high pressure.

### RELATED APPLICATIONS

This application claims benefit under 35 U.S.C. 119(e) of U.S. Provisional Application 62/393,284 filed Sep. 12, 2016, the disclosure of which is incorporated herein by reference.

### TECHNICAL FIELD

Embodiments of the disclosure relate to apparatus for burning or vaporizing plant matter and collection of the resulting smoke or vapor.

### BACKGROUND

Various plants have been found to contain compounds having pharmaceutical, therapeutic and/or cosmetic properties. Historically, certain compounds found in plants have been extracted for human administration through burning (also referred to as smoking) or vaporizing. Smoking of plant matter is typically achieved through rolling the plant matter into a cigarette and ignition of the cigarette, or through using a smoking apparatus such as a water pipe. Vaporization of plant matter is typically achieved through use of a vaporizer configured to contain a discreet amount of plant matter which is heated by air that is sufficiently hot to vaporize the compound but not cause the plant matter to ignite, and the hot air comprising the vaporized compound is then cooled and subsequently inhaled by the user.

Compositions for pharmaceutical, therapeutic and/or cosmetic use comprising compounds from plant matter may be prepared from plants using various methods using including solvent extraction and distillation.

### SUMMARY

Embodiments of the disclosure relate to a plant compound vaporization apparatus (PCVA) for heating plant matter to form vaporized plant compounds (“plant compound vapor”). The plant compound vapor is optionally comprised in smoke formed through ignition and burning of the plant matter or is produced without smoke through “vaporization” of the plant matter, in which the plant matter is heated to a temperature at which at least a portion of chemical constituents of the plant matter is vaporized but below a temperature at which the plant matter ignites. Optionally, the apparatus may be configured to provide burning or vaporization in a continuous manner, obviating the need to interrupt the burning or vaporization process in order to add plant matter to the apparatus. The apparatus may be configured to collect the plant compound vapor by introduction of the plant compound vapor into a solvent.

PCVA according to embodiments of the disclosure is advantageous relative to devices known in the prior art for burning or vaporization of plant matter, which typically use cigarettes.

According to an embodiment of the disclosure, the PCVA apparatus comprises a plant matter receptacle for containing plant matter, and a heating region for exposing the plant matter to heat through exposure to a heating element or causing the plant matter to ignite, and a plant matter conduit connecting the receptacle with the heating region to continuously convey, with a conveyance mechanism, plant matter from the receptacle to the heating region. Optionally, the plant matter is loose plant matter, and the conveyance mechanism may comprise, by way of example, a screw-driven conveyor, a conveyer belt, a tube-chain conveyor, a vibrating conveyor. Optionally, the plant matter is a compacted mass of plant matter, and the conveyance mechanisms comprises a mechanical drive or piston operable to move the compacted mass of plant matter. The BVA further comprises a vapor conduit located at a distance from the heating region, from which the plant compound vapor is removed from the environment of the plant matter. The distance between the heating region and the vapor conduit may remain constant, within a set of parameters, during the continuous burning or vaporization of the plant matter, thereby ensuring uniform consistency of the plant compound vapor.

The plant compound vapor, once obtained, may be analyzed using various methods to identify or quantify chemical components of the smoke. Alternatively, the plant compound vapor may be used for the manufacture of a compositions by solubilizing at least one compound comprised in the plant compound vapor into an appropriate solvent.

In an embodiment of the disclosure, the vapor conduit conveys the plant compound vapor to a solvent combination apparatus (SCA) to be combined with the solvent such that at least a portion of constituents in the pant compound vapor becomes dissolved in the solvent. In an embodiment of the disclosure, the SCA comprises a continuation of the vapor conduit configured to flow-connect the vapor conduit from the PCVA to a junction, and further comprises a solvent conduit that flow-connects a solvent receptacle to the junction, such that plant compound vapor generated in the PCVA is combined with the solvent at the junction to produce a vapor-solvent mix. Optionally, the vapor-solvent mix is flowed from the function through a nozzle to promote dissolution of the pant compound vapor in the solvent, by way of example through creating regions of high pressure. Optionally, the junction is flow-connected to the solvent receptacle, such that the solvent is circulated from the solvent receptacle, through the junction to mix with the pant compound vapor, and back into the solvent receptacle.

The solvent may be an alcoholic solvent or an aqueous solvent. Optionally, the alcohol is ethanol. According to an embodiment of the disclosure, the solvent may be an aqueous solution comprising at least one binding protein or a portion thereof characterized by being soluble in water or saline solution at room temperature or body temperature and comprising at least one region having an affinity to hydrophobic molecules. Optionally, the binding protein is a serum protein or a recombinantly produced equivalent thereof.

According to an embodiment of the aforementioned procedure of combining a plant compound vapor with a solvent which is then flowed through a nozzle to promote dissolution of the plant compound vapor in the solvent, may be used for chemical absorptions of pollution-related smoke or vapor from industrial exhaust streams.

PCVAs are advantageous in that they can be used to prepare uniform plant compound vapor from plant matter allowing for consistent, reproducible results each time the PCVA is used under the same conditions.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF FIGURES

Non-limiting examples of embodiments are described below with reference to figures attached hereto that are listed following this paragraph. Identical structures, elements or parts that appear in more than one figure are generally labeled with a same numeral in all the figures in which they appear, and a numeral labeling an icon representing a given feature in a figure may be used to reference the given feature. Dimensions of components and features shown in the figures are chosen for convenience and clarity of presentation and are not necessarily shown to scale.

FIG. 1 depicts a prior art apparatus for obtaining plant compound vapor of plant matter using a cigarette;

FIG. 2A depicts a cross sectional view of a plant matter burning apparatus and a SCA according to embodiments of the disclosure;

FIG. 2B depicts a nozzle used in conjunction with a SCA for combination of plant compound vapor with a solvent;

FIG. 2C depicts a cross sectional view of a plant matter vaporization apparatus according to embodiments of the disclosure; and

FIG. 2D depicts a cross sectional view of an alternative plant matter burning apparatus according to embodiments of the disclosure.

### DETAILED DESCRIPTION

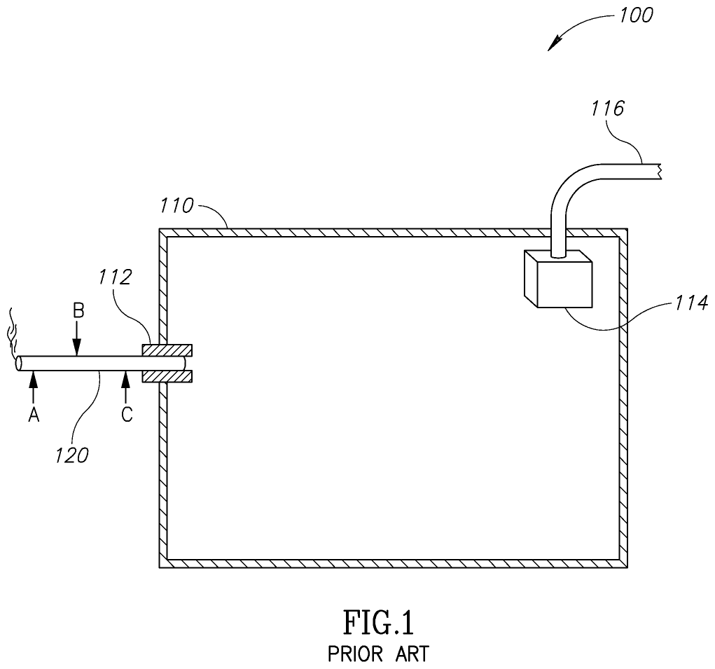

Reference is made to FIG. 1A, depicting a prior art smoking device 100 for obtaining smoke of plant matter using a cigarette. Smoking device 100 comprises a chamber 110, a cigarette holder 112, an air pump 114 and a smoke conduit 116. During operation of smoking device 100, a cigarette 120 may be inserted into cigarette holder 112 and ignited at its distal end (A). Chamber 110 may be a closed chamber having no other air inlet aside from smoke conduit 116. Air pump 114 may be configured to withdraw air from chamber 110 via smoke conduit 116 to outside of chamber 110, thereby creating a vacuum in chamber 110 and introducing smoke from ignited cigarette 120. At ignition, smoke travels from distal end through cigarette 120 and into chamber 110. Smoke may be analyzed or used within chamber 110. Optionally, smoke may be conveyed from chamber 110 via air pump 114 and smoke conduit 116 for further use or analysis.

A disadvantage of prior art smoking device 100 is that plant matter must be rolled into cigarettes in order to burn them, and loose plant matter cannot be used. An additional disadvantage is that cigarettes must be manually placed into cigarette holder 112 and the cigarette butt must then be removed from cigarette holder 112. Although a smoking device 100 may comprise multiple cigarette holders, in which cigarettes may be ignited in series in order to continuously provide smoke, cigarettes must be inserted and butts must be removed from the cigarette holders.

Another disadvantage of the smoking device 100 relates to the uniformity of smoke obtained. When a cigarette 120 is inserted into cigarette holder 112 and ignited, the smoke travels from point A on the cigarette, to point C, then into chamber 110. As the smoke travels, it is cooled by the plant matter located between point A and point C. Plant matter smoke typically comprises compounds having varying volatility. As the smoke cools, less volatile compounds are condensed upon the plant matter and more volatile compounds proceed through the cigarette into chamber 110. However, when cigarette 120 burns down to point B, the distance that the smoke generated has to travel is from point B to point C, which is a shorter distance than from point A to point C. As a result, a larger fraction of less volatile compounds enter the chamber 110 than when the cigarette 120 burns at point A. This leads to a lack of uniformity of smoke in chamber 110, as the nature of the smoke at a given time depends upon the length of the cigarette and the region of the cigarette being burnt at a given time.

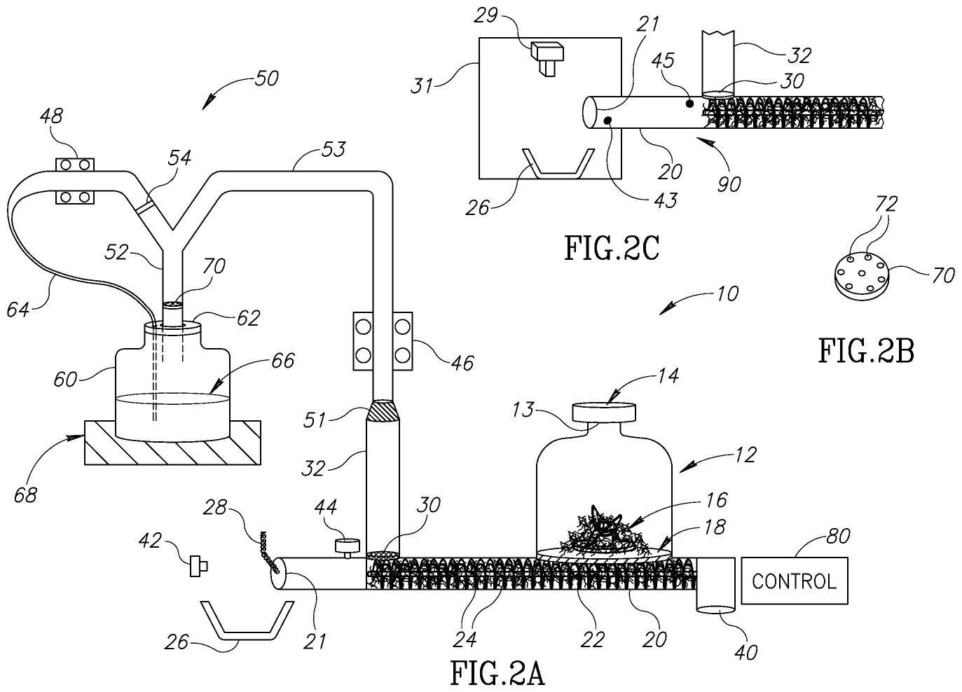

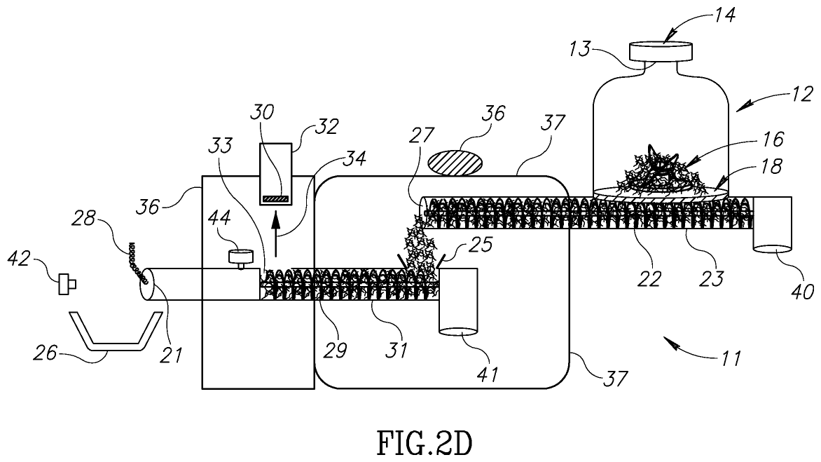

Reference is now made to FIG. 2A, depicts a cross sectional view of a plant matter burning apparatus 10 according to embodiments of the disclosure. Burning apparatus 10 comprises a plant receptacle 12 having an opening 13 and a cover 14. Plant receptacle 12 comprises an agitator 16 which is configured to rotate, thereby agitating loose plant matter. Plant receptacle 12, at is lower end, is connected to a plant conduit 20, and comprises a screen 18 which allows plant matter to pass through it into plant conduit 20.

Plant conduit 20 is operable to continuously convey plant matter from receptacle 12 to igniter 28. Plant conduit 20 is optionally fitted with a rotatable screw 22 having threads 24. At its proximal end, screw 22 is connected to motor 40 which rotates screw 22. Plant conduit 20 has an open distal end 21 in which direction plant matter is transported by screw 22 upon rotation. Distal end 21 is configured to lie above an ash collector 26. At distal end 21, an igniter 28 is positioned to ignite plant matter. Igniter 28 may comprise an electric wire which heats upon flowing electricity through the wire to a temperature sufficient to ignite plant matter.

Burning apparatus 10 comprises a vapor conduit 32 which is open at one end to plant conduit 20, and configured to allow flow of plant compound vapor from plant conduit 20. A screen 30 is placed between vapor conduit 32 and plant conduit 20 to prevent plant matter from entering vapor conduit 32 from plant conduit 20. vapor conduit 32 comprises a filter 51. Filter 51 is configured to allow flow of plant compound vapor but to prevent large particles (for example partially burnt plant matter or soot) from passing through.

Burning apparatus 10 comprises a sensor 42 configured to monitor ignition status of plant matter at distal end 21. Burning apparatus 10 further comprises a sensor 44 for determination of ignition status of plant matter within plant conduit 20 at a distance from distal end 21.

In an embodiment of the disclosure, burning apparatus 10 is connectable and/or operatively connected to solvent combination apparatus (SCA) 50. SCA 50 comprises a vapor conduit 53 that is flow-connected with vapor conduit 32 of burning apparatus 10. Vapor conduit 53 is configured with a pump 46 for creating a vacuum and drawing air from plant conduit 20 through vapor conduit 32. Pump 46 may be a peristaltic pump. While FIG. 2A shows SCA 50 connected to vapor conduit 32 of burning apparatus 10, the present disclosure also includes embodiments where vapor conduit 53 of SCA 50 is connectable or flow-connected to vapor conduit 32 of vaporization unit 90, or alternatively to smoke conduit 116 of smoking device 100, or alternatively to any other source of plant-derived or non-plant-derived vapor or smoke.

SCA 50 further comprises a solvent container 60 for collecting and holding a solvent 66 and a solvent circulation conduit 64 configured with a pump 48 for drawing solvent 66 from solvent container 60 to a junction 52 where solvent circulation conduit 64 and vapor conduit 53 are flow-connected with each other.

In an embodiment, junction 52 is shaped as a T-shaped or Y-shaped junction connecting solvent circulation conduit 64, vapor conduit 53, and solvent container 60, such that once solvent 66 and plant compound vapor from burning apparatus 10 combine at the junction to form a vapor-solvent mixture, the vapor-solvent mixture is directed back to solvent container 60 via an opening in solvent container cap 62. Recycling of solvent 66 is performed through action of pump 48.

In an embodiment of the disclosure, nozzle 70 is placed downstream of junction 52 and upstream of solvent container 60 in order to assists in dissolution of vaporized compounds (including but not limited to cannabinoids) comprised in the plant compound vapor transmitted through vapor conduit 53 into solvent 66 transmitted through solvent circulation conduit 64. In an embodiment of the disclosure, nozzle 70 is shaped to create areas of increased pressure for the vapor-and-solvent mixture to pass through.

Reference is now made to FIG. 2B showing an enlarged view of nozzle 70. Nozzle 70 comprises apertures 72 through which solvent can be pumped, forming areas of increased pressure on the solvent, thereby assisting in dissolution of smoke components into the solvent within the vapor-and-solvent mixture.

In an embodiment of the disclosure, solvent 66 comprises an organic solvent. Optionally, the organic solvent comprises of one or more of an acetonitrile, propylene glycol, a glycerol, DMSO, or an alcohol. Optionally, solvent 66 is a hydroalcoholic solvent comprising an alcohol and water. Optionally, the hydroalcoholic solvent comprises the alcohol at a concentration of between 5% and 99%, between 15% and 30%, about 20%. Optionally, the alcohol is ethanol. In an embodiment of the disclosure, the solvent is an aqueous solution comprising at least one binding protein or a portion thereof characterized by being soluble in water or saline solution at room temperature or body temperature and comprising a region having an affinity to hydrophobic molecules. Optionally, the binding protein is a serum protein, or a recombinantly produced equivalent thereof. Optionally, the binding protein comprises one of or a combination of more than one of the group consisting of: an albumin, a lipoprotein, a glycoprotein, an α globulin, a β globulin, and a γ globulin. Optionally, the albumin is selected from the group consisting of: a human serum albumin (HSA), a bovine serum albumin (BSA) or egg albumin (OVA).

Returning to FIG. 2A, cooling jacket 68 may be configured to surround solvent container 60, thereby cooling solvent 66 in solvent container 60. Operation of plant burning apparatus 10 and optionally SCA 50 is controlled by controller 80.

During the operation of burning apparatus 10, plant matter is introduced into plant receptacle 12 through opening 13 after removal of cover 14. Cover 14 is replaced and agitator 16 rotates to agitate plant matter, causing it to drop into plant conduit 20 via screen 18. Motor 40 rotates screw 22, thereby pushing plant matter located between threads 24 of screw 22 in the direction of distal end 21. Igniter 28 ignites plant matter at distal end 21. Burning is detected via sensor 42 or sensor 44, which optionally comprises one or more of: a temperature detector, an infrared sensor, and a smoke detector. Sensor 44 indicates if burning of plant matter occurs within a predetermined distance from distal end 21.

Sensors 42 and 44, igniter 28, agitator 16, and motor 40 are each connected to controller 80. The connections to sensors 42 and 44, igniter 28, agitator 16 and motor 40 may be wired or wireless. Controller 80 is configured to any one or a combination of one or more of igniter 28, agitator 16, and motor 40, to ensure consistent burning at distal end 21. Optionally, consistent burning is characterized by one or a combination of a consistent burning temperature, a consistent rate of smoke production, a consistent rate of mass of plant matter burned per unit time, or consistent amount of plant matter between the burning plant matter and vapor conduit 30. If no burning (or insufficient burning) is detected, controller may decrease rotation speed of motor 40, pushing less plant matter out of distal end 21, or controller may ignite igniter 28, ensuring burning (or more intense burning) of plant matter at distal end 21. If burning is detected within plant conduit 20 by sensor 44, controller 80 may increase speed of rotation of screw 22, thereby moving plant matter towards distal end 21 at a faster rate and ensuring burning of plant matter in region of distal end 21.

As plant matter is burnt at distal end 21, pump 46 acts to create a vacuum, drawing plant compound vapor from burnt plant matter at distal end 21 through plant conduit 20, and into vapor conduit 32. Plant compound vapor is then conveyed to junction 52 in SCA 50.

Solvent 66 is recirculated by action of pump 48, from solvent container 60 via solvent conduit 64 to junction 52. At junction 52, solvent joins with plant compound vapor from vapor conduit 32 and components of the plant compound vapor dissolve into solvent flowing through nozzle 70. Filter 54 removes solid impurities present within solvent 66.

Pumps 46 and 48 are controlled by controller 80. Pumps 46 and 48 may be configured to terminate operation if no plant compound vapor is being formed, as determined by sensor 42. Pumps 46 and 48 may be configured so that if pumping of pump 46 terminates, pump 48 terminates as well. This prevents the continued circulation of solvent 66, preventing solvent 66 from entering vapor conduit 32. While pump 46 is operating, solvent does not enter conduit 32 due to the pressure formed by pumping action of pump 46 on junction 52.

An advantage of burning apparatus 10 is the uniformity of plant compound vapor formed when burning plant matter, as the distance traveled by the heated plant compound vapor through the plant matter between distal end 21 and screen 30 remains constant, unlike the prior art apparatus described above with reference to FIG. 1.

Reference is now made to FIG. 2C which depicts a cross sectional view of a plant matter vaporization unit 90 according to embodiments of the disclosure. Vaporization unit 90 may be used in conjunction with parts of burning apparatus 10, if vaporization of plant matter is desired, as opposed to burning.

Vaporization unit 90 comprises a vaporization chamber 31, temperature sensors 43 and 45, and heater 29. Operation of vaporization unit 90 serves to vaporize compounds comprised in plant matter 16 and collect the resulting vapor.

During operation of vaporization unit 90, plant matter is advanced to distal end 21 of plant conduit 20 by rotation of screw 22. Heater 29 heats plant matter at distal end 21 to a temperature at which plant vapor is formed. Screw 22 is configured to advance plant matter at a rate allowing for vaporization of plant matter at distal end 21. After formation of vapor, vapor is drawn through plant matter in plant conduit 20, through screen 30 and into conduit 32. The vapor may be processed by combining with a solvent as described with reference to dissolution of plant compound vapor in FIG. 2A. The spent plant matter falls to ash collector 26 as plant matter advances in the direction of distal end 21. Vaporization chamber 31 may be an enclosed chamber which maintains the vapor within while allowing vapor to be drawn through plant conduit 20.

Temperature sensors 43 and 45 and heater 29 are connected to controller 80 (shown in FIG. 2A) and are configured to maintain temperature of the plant matter at distal end 21 at a predetermined temperature. If temperature is lower than a predetermined temperature at sensor 43, controller 80 may signal to heater 29 to increase temperature. If temperature of the plant matter at sensor 45 is higher than a predetermined temperature, controller 80 may signal to heater 29 to decrease temperature.

According to an embodiment of the disclosure, distance between threads 24 may vary across the length of screw 22. For example, toward distal end 21, the distance between threads 24 may be increased toward distal end 21.

Reference is now made to FIG. 2D depicting a cross sectional view of a plant matter burning apparatus 11 according to embodiments of the disclosure. Burning apparatus 11 comprises a plant receptacle 12 having an opening 13 and a cover 14. Plant receptacle 12 comprises an agitator 16 which is configured to rotate, thereby agitating plant matter. Plant receptacle, at is lower end, is connected to a plant conduit 23, and comprises a screen 18 which allows plant matter to pass through it into plant conduit 20.

Plant conduit 23 is fitted with a rotatable screw 22. At its proximal end, screw 22 is connected to motor 40 which rotates screw 22. Plant conduit 23 has an open distal end 27 in which direction plant matter is transported by screw 22 upon rotation. Distal end 27 is configured to convey plant matter to container 25. Container 25 is fitted with a screw 29, rotatable by a motor 41. Screw 29 feeds plant matter into a plant conduit 31. Plant conduit 31 has a distal end 21.

At distal end 21, an igniter 28 is positioned to ignite plant matter. Igniter 28 may comprise an electric wire which heats upon flowing electricity through the wire to a temperature sufficient to ignite plant matter. Burning apparatus 11 comprises a vapor conduit 32 which is open at one end to container 25, and configured to allow flow of plant compound vapor produced at distal end 21, through container 25

During the operation of burning apparatus 11, motor 40 rotates screw 22 to advance plant matter through plant conduit 23 towards distal end 27. From distal end 27, plant matter enters into container 25, where it is conveyed by screw 29. A vacuum is operated on vapor conduit 32 to create a vacuum in container 25. This prevents heat and smoke from combustion occurring at distal end 21 from heating plant matter located in plant conduit 23.

In an embodiment of the disclosure, container 25 and optionally a portion of plant conduit 31 is enclosed within an enclosure 37. The enclosure may have an opening 35 to allow access into container 25. Opening 35 may be covered with a cover 36, for example during operation of burning apparatus 11, in order to prevent leakage of air into the enclosure during vacuum operation of vapor conduit 32.

In an embodiment of the disclosure, burning apparatus 11 may be configured to have a gap, schematically indicated by arrow 34, between an opening 33 in plant conduit 31 and vapor conduit 32. Opening 33 and a portion of the vapor conduit may be enclosed within an enclosure 38, so that plant compound vapor produced in distal end 21 will be efficiently captured through vacuum operation of vapor conduit 32 and traverse gap 34 between opening 33 and vapor conduit 32. The presence of gap 34 may advantageously reduce or prevent plant particles from reaching vapor conduit 32.

While FIGS. 2A-2D shows plant conduit 20 conveying loose plant matter through a screw-driven conveyance mechanism, plant conduit 20 optionally comprises, alternatively or additionally, one or more alternative conveyance mechanism (not shown) for conveying loose plant matter, by way of example, a conveyer belt, a tube-chain conveyor, or a vibrating conveyor. Optionally, plant conduit 20 comprises a mechanical drive or piston operable to move a compacted mass (now shown) of plant matter towards igniter 28. The compacted mass of plant matter may be in a form of, for example, a cigar or a cigarette.

There is therefore provided in the present disclosure an apparatus for burning or vaporization of plant material, the apparatus comprising: a heating element configured to heat the plant matter to a temperature at which at least a portion of chemical constituents of the plant matter is vaporized to form a vapor; a plant matter conduit comprising a conveyance mechanism operable to continuously convey plant matter to the heating element; and a vapor conduit, flow-connected to the plant matter conduit, configured to withdraw the vapor generated by the heating of the plant matter by the heating element, from the plant matter conduit. In an embodiment, the plant matter conduit is configured to convey loose plant matter. Optionally, the conveyance mechanism is one or a combination of more than one from the group consisting of: a screw-driven mechanism, a conveyer belt, a tube-chain conveyor, or a vibrating conveyor. Optionally, the apparatus comprises a plant matter receptacle for containing loose plant matter, wherein the plant matter conduit is configured to convey plant matter from the plant matter receptacle to the heating element. Optionally, the plant matter conduit is configured to convey compacted plant matter, wherein the conveyance mechanism comprises a mechanical drive or a piston operable to move the compacted mass towards the heating element.

In an embodiment, the apparatus comprises at least one sensor is configured to monitor heating status of the plant matter by the heating element. Optionally, the at least one sensor comprises one or more of: a temperature detector, an infrared sensor, and a smoke detector. Optionally, the apparatus comprises a controller operable to control heating by the heating element and/or conveyance of the plant matter by the plant matter conduit responsive to the at least one sensor to ensure consistent heating of the plant matter. Optionally, the consistent heating is one or more selected from: consistent heating temperature, consistent smoke production, and consistent rate of mass of the plant matter heated or burned per unit time.

In an embodiment, the heating element is operable to heat the plant matter to a temperature at which at least a portion of chemical constituents of the plant matter is vaporized but below a temperature at which the plant matter ignites. Optionally, the heating element is an igniter operable to ignite the plant matter.

There is also provided in the disclosure an apparatus for burning or vaporization of plant material, the apparatus comprising: a heating element configured to heat the plant matter to a temperature at which at least a portion of chemical constituents of the plant matter is vaporized to form a vapor; a vapor conduit, flow-connected to the plant matter conduit, configured to withdraw a vapor generated by the heating of the plant matter; and a solvent conduit flow-connected to the vapor conduit at a junction, the solvent conduit being configured to circulate solvent, such that vapor withdrawn from the plant matter conduit contacts the circulated solvent.

In an embodiment, the apparatus comprises a nozzle flow-connected to the vapor conduit, wherein the nozzle is configured to crease regions of high pressure for the vapor and/or solvent traversing the nozzle. Optionally, the nozzle is placed downstream of the junction.

In an embodiment, the apparatus further comprises the solvent. Optionally, the solvent comprises one or a combination of more than one of an organic solvent selected from the group consisting of an acetonitrile, propylene glycol, a glycerol, DMSO, or an alcohol. Optionally, the solvent consists of an organic solvent. Optionally, the solvent is a hydroalcoholic solvent comprising an alcohol and water. Optionally, the alcohol is ethanol. Optionally, the solvent comprises an aqueous solution comprising a binding protein or a portion thereof characterized by being soluble in water or saline solution at room temperature or body temperature and comprising at least one region having an affinity to hydrophobic molecules. Optionally, the binding protein is a serum protein or a recombinantly produced equivalent thereof. Optionally, the binding protein comprises one of or a combination of more than one of the group consisting of: an albumin, a lipoprotein, a glycoprotein, an α globulin, a β globulin, and a γ globulin.

In the description and claims of the present application, each of the verbs, “comprise,” “include” and “have,” and conjugates thereof, are used to indicate that the object or objects of the verb are not necessarily a complete listing of components, elements or parts of the subject or subjects of the verb.

Descriptions of embodiments of the disclosure in the present application are provided by way of example and are not intended to limit the scope of the invention. The described embodiments comprise different features, not all of which are required in all embodiments of the disclosure. Some embodiments utilize only some of the features or possible combinations of the features. Variations of embodiments of the disclosure that are described, and embodiments of the disclosure comprising different combinations of features noted in the described embodiments, will occur to persons of the art. The scope of the invention is limited only by the claims.

1. An apparatus for burning or vaporization of plant material, the apparatus comprising:
a heating element configured to heat the plant matter to a temperature at which at least a portion of chemical constituents of the plant matter is vaporized to form a vapor;
a plant matter conduit comprising a conveyance mechanism operable to continuously convey plant matter to the heating element; and
a vapor conduit, flow-connected to the plant matter conduit, configured to withdraw the vapor generated by the heating of the plant matter by the heating element, from the plant matter conduit.

2. The apparatus according to claim 1, wherein the plant matter conduit is configured to convey loose plant matter.

3. The apparatus according to claim 2, wherein the conveyance mechanism is one or a combination of more than one from the group consisting of: a screw-driven mechanism, a conveyer belt, a tube-chain conveyor, or a vibrating conveyor.

4. The apparatus according to claim 2, further comprising a plant matter receptacle for containing loose plant matter, wherein the plant matter conduit is configured to convey plant matter from the plant matter receptacle to the heating element.

5. The apparatus according to claim 1, wherein the plant matter conduit is configured to convey compacted plant matter, wherein the conveyance mechanism comprises a mechanical drive or a piston operable to move the compacted mass towards the heating element.

6. The apparatus according to claim 1, further comprising at least one sensor configured to monitor heating status of the plant matter by the heating element.

7. The apparatus according to claim 6, wherein the at least one sensor comprises one or more of: a temperature detector, an infrared sensor, and a smoke detector.

8. The apparatus according to claim 6, further comprising a controller operable to control heating by the heating element and/or conveyance of the plant matter by the plant matter conduit responsive to the at least one sensor to ensure consistent heating of the plant matter.

9. The apparatus according to claim 8, wherein consistent heating is one or more selected from: consistent heating temperature, consistent smoke production, and consistent rate of mass of the plant matter heated or burned per unit time.

10. The apparatus according to claim 1, wherein the heating element is operable to heat the plant matter to a temperature at which at least a portion of chemical constituents of the plant matter is vaporized but below a temperature at which the plant matter ignites.

11. The apparatus according to claim 1, wherein the heating element is an igniter operable to ignite the plant matter.

12. An apparatus for burning or vaporization of plant material, the apparatus comprising:
a heating element configured to heat the plant matter to a temperature at which at least a portion of chemical constituents of the plant matter is vaporized to form a vapor;
a vapor conduit, flow-connected to the plant matter conduit, configured to withdraw a vapor generated by the heating of the plant matter; and
a solvent conduit flow-connected to the vapor conduit at a junction, the solvent conduit being configured to circulate solvent, such that vapor withdrawn from the plant matter conduit contacts the circulated solvent.

13. The apparatus according to claim 12, further comprising a nozzle flow-connected to the vapor conduit, wherein the nozzle is configured to crease regions of high pressure for the vapor and/or solvent traversing the nozzle.

14. The apparatus according to claim 12, wherein the nozzle is placed downstream of the junction.

15. The apparatus according to claim 12, further comprising the solvent.

16. The apparatus according to claim 15, wherein the solvent comprises one or a combination of more than one of an organic solvent selected from the group consisting of an acetonitrile, propylene glycol, a glycerol, DMSO, or an alcohol.

17. The apparatus according to claim 16, wherein the solvent consists of an organic solvent.

18. The apparatus according to claim 16, wherein the solvent is a hydroalcoholic solvent comprising an alcohol and water.

19. The apparatus according to claim 16, wherein the alcohol is ethanol.

20. The apparatus according to claim 15, wherein the solvent comprises an aqueous solution comprising a binding protein or a portion thereof characterized by being soluble in water or saline solution at room temperature or body temperature and comprising at least one region having an affinity to hydrophobic molecules.

21. The apparatus according to claim 20, wherein the binding protein is a serum protein or a recombinantly produced equivalent thereof.

22. The apparatus according to claim 21, wherein the binding protein comprises one of or a combination of more than one of the group consisting of: an albumin, a lipoprotein, a glycoprotein, an α globulin, a β globulin, and a γ globulin.
